(19) United States
(12) Patent Application Publication
(10) Pub. No.: US 2023/0029162 A1
(43) Pub. Date: Jan. 26, 2023

(54) HEAT GENERATOR FOR KNEES

(71) Applicant: Kobayashi Pharmaceutical Co., Ltd., Osaka-shi (JP)

(72) Inventor: Kouta Nakabayashi, Ibaraki-shi (JP)

(73) Assignee: Kobayashi Pharmaceutical Co., Ltd., Osaka-shi (JP)

(21) Appl. No.: 17/788,961

(22) PCT Filed: Dec. 2, 2020
(86) PCT No.: PCT/JP2020/044856

§ 371 Date: Jun. 24, 2022

(30) Foreign Application Priority Data
Dec. 27, 2019 (JP) 2019-239561

(51) Int. Cl. A41D 13/005; A41D 13/06; A61F 7/03

(52) U.S. Cl. CPC A41D 13/005; A41D 13/06; A61F 7/03

(57) ABSTRACT

Provided is a heat generator for knees capable of effectively warming a knee joint. A heat generator for knees 1 includes a heat-generating material and a pouch that is formed from a pair of sheets and that accommodates the heat-generating material inside. The pouch includes a peripheral joint portion that is formed from the pair of sheets that are joined, and that has a heat generation area inside, and a partition portion that divides the heat generation area into sections in the left-and-right direction by the pair of sheets joined in the up-and-down direction on the inner side of the peripheral joint portion.

### TECHNICAL FIELD

The present invention relates to a heat generator for knees.

### BACKGROUND ART

Warming is known to alleviate pain caused by inflammation of the knee such as with knee osteoarthritis. For example, Patent Literature 1 discloses a thermal treatment device that secures a chemical warmer to the knee with a holder.

The thermal treatment device disclosed in Patent Literature 1 includes a cylindrical holder to be attached to the knee and a chemical warmer. The holder has a pocket-shaped chemical-warmer storage portion that stores the chemical warmer. This allows the chemical warmer to be applied to the knee, and a user can warm the affected area.

### CITATION LIST

### Patent Literature

Patent Literature 1: JP2011-212462A

### SUMMARY OF INVENTION

### Technical Problem

However, due to the holder present between the chemical warmer and the knee, the thermal treatment device disclosed in Patent Literature 1 decreases the heat transmitted from the chemical warmer to the affected area.

Because inflammation at the knee joint occurs in the synovium or fat tissue, it is more effective to warm the knee from the lateral sides rather than the front side, in which the patella is located. The chemical warmer storage portion located on the front side of the knee conducts the heat from the chemical warmer through the patella to warm the affected area. Thus, the heat transferred to the affected area further decreases.

Given the circumstances above, the present invention was made, and an object of the invention is to provide a heat generator for knees capable of effectively warming the knee joint.

### Solution to Problem

A heat generator for knees according to an embodiment of the present invention includes a heat-generating material and a pouch that is formed from a pair of sheets and that accommodates the heat-generating material inside. The pouch includes a peripheral joint portion that is formed from the pair of sheets that are joined, and that includes a heat generation area inside, and a partition portion that divides the heat generation area into sections in the left-and-right direction by the pair of sheets joined in the up-and-down direction on the inner side of the peripheral joint portion.

The partition portion of the heat generator for knees is preferably positioned in the center portion between the right end and the left end of the heat generation area in the left-and-right direction.

The upper edge of the heat generation area in the heat generator for knees is preferably formed so as to be positioned lower as the upper edge of the heat generation area approaches the partition portion in the left-and-right direction.

The upper edge of the pouch in the heat generator for knees is preferably formed so as to be positioned lower as the upper edge of the pouch approaches the partition portion in the left-and-right direction.

The heat generator for knees preferably further includes a belt configured to secure the pouch on a user's leg.

The pouch of the heat generator for knees is preferably formed so as to be symmetrical in the up-and-down direction.

### Advantageous Effects of Invention

The heat generator for knees according to the present invention in the embodiment above is advantageous in its capability of effectively warming the knee joint.

### BRIEF DESCRIPTION OF DRAWINGS

FIG. 1: FIG. 1 is a perspective view showing the heat generator for knees according to an embodiment of the present invention in use.

FIG. 2: FIG. 2 is a schematic cross-sectional view showing the structure of the knee joint.

FIG. 3: FIG. 3 is a front view of the heat generator for knees according to the embodiment above.

FIG. 4: FIG. 4 is a cross-sectional view taken along line X1-X1 in FIG. 3.

FIG. 5: FIG. 5(A) is a front view of a heat generator for knees according to modification example 1. FIG. 5(B) is a front view of a heat generator for knees according to modification example 2.

FIG. 5(C) is a front view of a heat generator for knees according to modification example 3.

FIG. 6: FIG. 6(A) is a front view of a Comparative Example. FIG. 6(B) is a front view of Example 1. FIG. 6(C) is a front view of Example 2.

FIG. 7: FIG. 7 is a view showing the temperature distribution in thermographic images of Examples 1 and 2 and the Comparative Example.

### DESCRIPTION OF EMBODIMENTS

### (1) Embodiment

The following describes a heat generator for knees 1 according to an embodiment of the present invention in detail.

(1.1) Outline

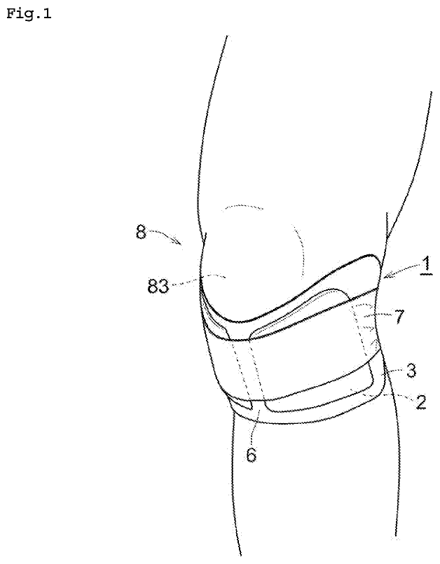

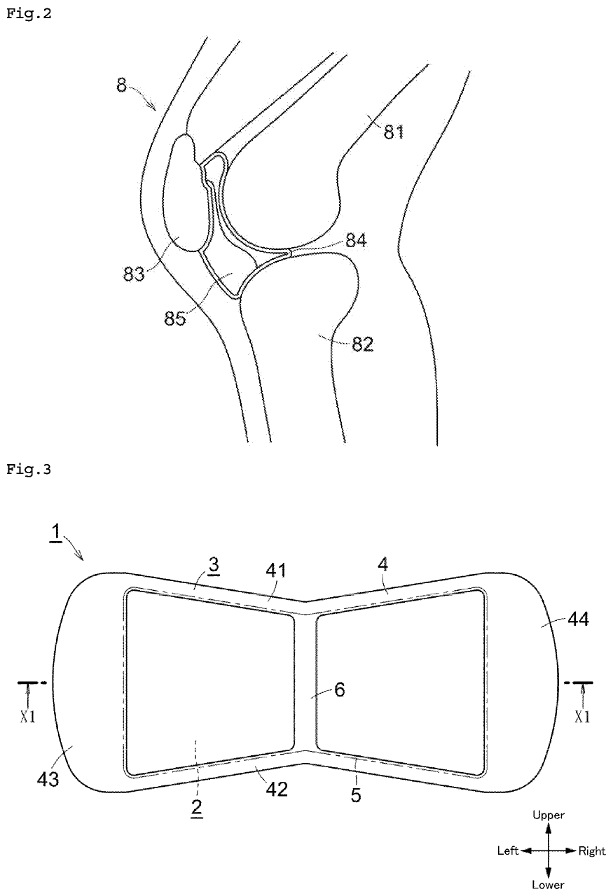

As shown in FIG. 1, the heat generator for knees 1 is a heat generator for warming a user's knee 8 to alleviate the pain in the knee 8. The cause of the pain of the knee 8 is, for example, osteoarthritis of the knee. Osteoarthritis of the knee is an arthropathy in the knee joint, which wears the cartilage on the surface of the knee joint and deforms the meniscus, causing inflammation, for example, in the synovium 84 (FIG. 2), and further pain. As shown in FIG. 2, the knee joint is present between the femur 81 and the tibia 82 and between the femur 81 and the patella 83, and these are covered by the synovium 84.

One way to relieve pain caused by inflammation of the synovium 84 is to warm the area affected by inflammation. However, heat applied to the knee joint from the front warms the affected area through the patella 83, which is not effective in warming the affected area. Applying heat from both sides of the knee joint is more effective.

Given the fact above, the heat generator for knees 1 according to this embodiment is designed so that heat can be applied from both sides of the knee joint. The heat generator for knees 1 includes a heat-generating material 2, a pouch 3, and a belt 7 as shown in FIG. 1, and divides a heat generation area 5 of the pouch 3 with a partition portion 6 in the left-and-right direction as shown in FIG. 3.

Dividing the heat generation area 5 into sections in the left-and-right direction allows the high-temperature region in the heat generation area 5 to be located in the center portion of each section. Thus, for example, as shown in FIG. 1, placing the partition portion 6 below the part at which the patella 83 is located allows the heat generator to be attached to the knee 8, while the high-temperature region is applied to both sides of the knee joint. This effectively provides a warming effect to the affected part of the knee joint.

The following describes the heat generator for knees 1 according to this embodiment in more detail. For convenience of explanation, the description below will be given assuming the up-and-down direction of the heat generator for knees 1 worn by a standing user. Specifically, as shown in FIG. 3, the longitudinal direction of the partition portion 6 is defined as the “up-and-down direction,” and “upper” and “lower” are defined as indicated by arrows on the drawings. The direction perpendicular to the up-and-down direction is defined as the “left-and-right direction.” However, the definitions of these directions are not intended to limit the embodiments of use of the heat generator for knees 1.

(1.2) Details

(1.2.1) Heat-Generating Material

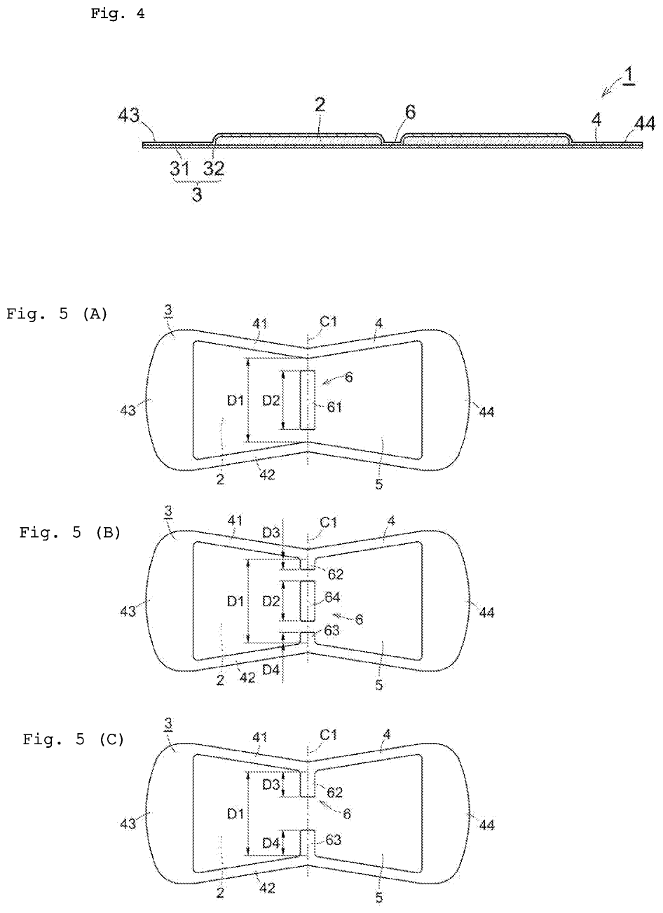

The heat-generating material 2 is a material that can generate heat. In this embodiment, the heat-generating material 2 comes into contact with air and undergoes oxidation, thus generating heat. The heat-generating material 2 is a powdery material and is enclosed in the pouch 3 as shown in FIG. 4.

Examples of the heat-generating material 2 include oxidizable metals, activated carbon, carbon black, water-retaining agents, metal salts (e.g., sodium chloride), and water. Oxidizable metals radiate heat of oxidation reaction, and examples are one, two, or more powders or fibers selected from the group consisting of iron, aluminum, zinc, manganese, magnesium, and calcium. Of these, iron powder is preferable from the standpoint of handling, safety, production costs, storage, and stability. Iron powder is, for example, one, two, or more members selected from the group consisting of reduced iron powder and atomized iron powder. Examples of water-retaining agents include wood flour, vermiculite, diatomaceous earth, perlite, silica gel, alumina, and water-absorbing resin. The heat-generating material 2 for use may be the composition used in conventional disposable body warmers as is, and is not particularly limited in the present invention.

The heat-generating material 2 for use may be a material other than those that generate heat by coming into contact with air. For example, the heat-generating material 2 for use may be a material that generates heat by being irradiated with microwaves of a microwave oven (e.g., ceramic powder such as ferrite and red beans).

(1.2.2) Pouch

The pouch 3 is a component formed in a bag shape and has storage space for accommodating the heat-generating material 2 inside. As shown in FIG. 4, the pouch 3 includes a front sheet 31 and a back sheet 32 that are superposed one above the other with the outer peripheral portion joined. (This joint portion is referred to as “peripheral joint portion 4” below.)

The pouch 3 for use is preferably a resin film from the standpoint of its flexibility, strength, and durability against heat generation of the heat-generating material 2. The resin for use in the resin film is preferably a thermoplastic resin. Examples of thermoplastic resins include polyethylene, polypropylene, polyester, polyamide, polyurethane, polystyrene, polyvinyl alcohol, polyvinyl chloride, polyvinylidene chloride, polycarbonate, and ethylene-vinyl acetate copolymers. These resins may be used singly or in a combination of two or more. The flexibility of the pouch 3 allows the heat generator for knees 1 to flexibly deform along the contour of the user's knee 8 when the heat generator is attached to the knee 8.

From the standpoint of achieving excellent texture to the skin, the front sheet 31 preferably has a woven or non-woven fabric on the outer side of the resin film. Examples of fiber materials for the woven or non-woven fabric include natural fibers, such as cotton, linen, silk, and paper; semisynthetic fibers, such as rayon and acetate; synthetic fibers, such as nylon, vinylon, polyester, acrylic, polyethylene, polypropylene, polyvinyl chloride, and polybutylene terephthalate; and mixed fibers of these fibers. Of these, from the standpoint of achieving excellent texture to the skin, the fiber material is preferably, for example, nylon, polyester, or polypropylene. These fiber materials may be used singly or in a combination of two or more. The basis weight of the woven or non-woven fabric is not particularly limited as long as the fabric can prevent the leakage of the heat-generating material 2 to the outside of the pouch 3. One example of the basis weight is 20 g/m² or more and 70 g/m² or less.

However, the pouch 3 in the present invention may not be a laminate formed of a resin film and a woven or non-woven fabric. The pouch 3 may be formed of a single resin film or a single woven or non-woven fabric.

The pouch 3 is breathable. Because the resin film is a porous resin film or has a plurality of pores formed on it (not shown), the pouch 3 has a plurality of pores and can breathe through the pores. The plurality of pores may be formed uniformly over an entire surface of the pouch 3, or may be formed densely in part of the surface. The diameter of each pore is not limited as long as the pore can prevent the heat-generating material 2 from leaking through the pore. An example of the pore diameter is 0.1 μm or more and 30 μm or less. Because the sensory temperature of the heat generator for knees 1 varies according to the permeability of the pouch 3, the size, the shape, and the number of pores are appropriately set taking into consideration the sensory temperature of the heat generator for knees 1 and the degree of preventing leakage of the heat-generating material 2. The pouch 3 formed from a single non-woven fabric may not have a plurality of pores formed in it.

The pouch 3 preferably has a predetermined moisture permeability. The pouch 3 preferably has a moisture permeability of 200 g/(m²·day) or more and 1000 g/(m²·day) or less, and more preferably 200 g/(m²·day) or more and 600 g/(m²·day) or less.

The peripheral joint portion 4 is achieved, for example, by welding (heat sealing). However, the joint portion in the present invention does not have to be made by welding, and may be achieved by, for example, adhesion, sewing, compression bonding, or a combination of these. As shown in FIG. 3, the peripheral joint portion 4 is continuous along the entire circumference of the pouch 3 and surrounds the heat generation area 5. The “heat generation area 5” as used here means one area including all the heat-generating sections in the heat generator for knees 1.

The peripheral joint portion 4 is formed along the circumference of the pouch 3. The peripheral joint portion 4 includes an upper joint portion 41 located above the storage space of the heat-generating material 2, a lower joint portion 42 located below the storage space of the heat-generating material 2, a left joint portion 43 located on the left side of the storage space of the heat-generating material 2, and a right joint portion 44 located on the right side of the storage space of the heat-generating material 2.

The upper joint portion 41 is part of the peripheral joint portion 4 and forms the upper edge of the heat generation area 5. The upper joint portion 41 is formed with a constant width over the entire length in the longitudinal direction. Its lower edge in the width direction constitutes the upper edge of the heat generation area 5 whereas its upper edge in the width direction constitutes the upper edge of the pouch 3. The upper joint portion 41 inclines with respect to the left-and-right direction so as to be positioned lower as the upper joint portion 41 approaches the partition portion 6 in the left-and-right direction. Specifically, the upper edge of the heat generation area 5 is formed so as to be positioned lower as the upper edge of the heat generation area 5 approaches the partition portion 6 in the left-and-right direction, and the upper edge of the pouch 3 is formed so as to be positioned lower as the upper edge of the pouch 3 approaches the partition portion 6 in the left-and-right direction.

Because the upper edge of the heat generation area 5 is formed so as to be positioned lower as the upper edge of the heat generation area 5 approaches the partition portion 6 in the left-and-right direction, the heat generator for knees 1 can be disposed to not be positioned in front of the patella 83, but positioned at both sides of the knee joint (the parts adjacent to the patella 83) when the heat generator for knees 1 is attached to the knee 8. Additionally, because the thick part of the heat generator for knees 1 due to the heat-generating material 2 does not reach the part of the knee at which the patella 83 is located, the movement of the knee is not hindered, for example, while walking.

Additionally, because the upper edge of the pouch 3 is formed so as to be positioned lower as the upper edge of the pouch 3 approaches the partition portion 6 in the left-and-right direction, the heat generator for knees 1 can be disposed such that the pouch 3 does not cover the part of the knee at which the patella 83 is located when the heat generator for knees 1 is attached to the knee 8. This can prevent displacement of the heat generator for knees 1 when the patella 83 moves during exercise bending the knee 8, such as walking or bending and stretching. Additionally, because the part of the knee at which the patella 83 is located is not covered not only by the pouch 3 but also by the thick part of the heat generator due to the heat-generating material 2, the movement of the knee is further not hindered, for example, during walking.

In this embodiment, the upper edge of the heat generation area 5 and the upper edge of the pouch 3 are formed by a straight line in the part on the left side of the partition portion 6 and the part on the right side of the partition portion 6. However, in the present invention, for example, the entire upper edge of the heat generation area 5 and/or the pouch 3 may be formed in an arc shape, or only the part proximal to the partition portion 6 may be formed in an arc shape, and the other remaining part may be formed by a straight line substantially parallel to the left-and-right direction.

The lower joint portion 42 is a portion of the peripheral joint portion 4 and forms the lower edge of the heat generation area 5. The lower joint portion 42 is formed with a constant width over the entire length in the longitudinal direction. Its upper edge in the width direction constitutes the lower edge of the heat generation area 5, and its lower edge in the width direction constitutes the lower edge of the pouch 3. The lower joint portion 42 is inclined with respect to the left-and-right direction so as to be positioned lower as the lower joint portion 42 approaches the partition portion 6 in the left-and-right direction. Thus, the pouch 3 is formed symmetrically with respect to the line that is along the left-and-right direction, and that passes through the center of the up-and-down direction of the pouch 3 (i.e., symmetrical in the up-and-down direction). This prevents a user from accidentally attaching the heat generator for knees 1 upside down.

The left joint portion 43 and the right joint portion 44 each connect one end portion of the upper joint portion 41 and one end portion of the lower joint portion 42. The left joint portion 43 and the right joint portion 44 are each formed so as to be wider toward the center in the up-and-down direction. The outer edge of the left joint portion 43 and the outer edge of the right joint portion 44 in the width direction are formed in an arc shape. However, the left joint portion 43 and the right joint portion 44 may be formed with a constant width in the up-and-down direction, and the outer edge may be formed in a straight line.

The heat generation area 5 may be of any size as long as the heat generation area 5 can apply heat to the knee joint. For example, the heat generation area 5 of the following size is preferable. The dimension in the up-and-down direction at both end portions in the left-and-right direction (see dimension H3 in FIG. 6(C)) is preferably, for example, 3 cm or more and 10 cm or less, more preferably 5 cm or more and 10 cm or less, and still more preferably 6 cm or more and 9.5 cm or less. The dimension in the up-and-down direction in the center portion of the left-and-right direction (see dimension H4 in FIG. 6(C)) is, for example, 1 cm or more and 7 cm or less, more preferably 3 cm or more and 7 cm or less, and still more preferably 4 cm or more and 6.5 cm or less.

The pouch 3 has the partition portion 6 formed on the inner side of the peripheral joint portion 4. The partition portion 6 divides the heat generation area 5 into sections in the left-and-right direction by the pair of sheets 31, 32 joined along the up-and-down direction. Dividing the heat generation area 5 into sections in the left-and-right direction allows the high-temperature region of each section to be apart from each other in the left-and-right direction, making the maximum temperature higher than that of heat generators with no partition portion 6. This allows the high-temperature regions to reach both sides of the knee joint when the heat generator for knees 1 is attached to the knee 8 and effectively provides a warming effect to appropriate parts.

The partition portion 6 is linearly formed in the up-and-down direction and connected to the lower joint portion 42 at the lower end and connected to the upper joint portion 41 at the upper end. The partition portion 6 is formed with a constant width over the entire length in the up-and-down direction. The width of the partition portion 6 is, for example, preferably 0.1 cm or more and 3 cm or less, more preferably 0.1 cm or more and 2.5 cm or less, and still more preferably 0.5 cm or more and 2 cm or less. In this embodiment, the partition portion 6 is achieved by welding (heat sealing), which joins a pair of sheets. However, in the present invention, the partition portion 6 does not have to be made by welding as long as a pair of sheets can be joined, and may be achieved, for example, by adhesion, sewing, compression bonding, or a combination of these.

The partition portion 6 is located in the center portion between the right end and the left end of the heat generation area 5 in the left-and-right direction. Thus, when the heat generator for knees 1 is attached to the knee such that the partition portion 6 is located at the part below the patella 83, the high-temperature region of each section reaches each side of the knee joint. Additionally, because the partition portion 6 is more easily bent than the part filled with the heat-generating material 2, the heat generator for knees 1 attached to the knee 8 easily fits the knee.

The “center portion” as used here may be any position that includes the center line between the right end and the left end of the heat generation area 5. For example, if the partition portion 6 is off the center line by the width of the partition portion 6, this can also be considered to be located in the center portion between the right end and the left end of the heat generation area 5.

(1.2.3) Belt

The belt 7 secures the pouch 3 to a leg of a user as shown in FIG. 1. This allows the heat generation area 5 of the heat generator for knees 1 to remain in contact with both sides of the knee joint. The belt 7 includes attachment parts formed at both ends in the longitudinal direction (not shown). While one attachment part is secured to the pouch 3 with the pouch 3 applied to the knee 8, the belt 7 is wound around a leg, and the other attachment part is secured to a portion of the belt 7 in the longitudinal direction. This secures the pouch 3 to the leg and attaches the heat generator for knees 1 to the knee 8.

Examples of attachment parts include hook-and-loop fasteners, hooks, buttons, zippers, and double-sided tape. In this embodiment, a hook-and-loop fastener is used. Although the belt 7 is formed so as to be removable from the pouch 3, the belt 7 may also be integrally formed with the pouch 3.

(2) Action Effect

As described above, the heat generator for knees 1 according to this embodiment has the heat generation area 5 divided into sections by the partition portion 6. Thus, attaching the heat generator for knees 1 such that the partition portion 6 is located below the patella 83 allows the high-temperature regions within the temperature distribution of the heat generation area 5 to be applied to the right and left sides of the knee joint. Thus, the heat generator for knees 1 according to this embodiment can effectively warm the knee joint. Additionally, because the partition portion 6 is more easily bent than the part filled with the heat-generating material 2, the heat generator for knees 1 attached to the knee 8 easily fits the knee 8.

Because the partition portion 6 is located in the center portion of the heat generation area 5, the high-temperature region of the heat generation area 5 is divided into substantially symmetrical sections on the right and left sides with respect to the center portion and can effectively warm both sides of the knee joint.

The upper edge of the heat generation area 5 is formed so as to be positioned lower as the upper edge of the heat generation area 5 approaches the partition portion 6 in the left-and-right direction. This prevents the part filled with the heat-generating material 2, which is thicker than the part without the heat-generating material 2, from covering the part in which the patella 83 is located to thereby decrease thermal efficiency.

The upper edge of the pouch 3 is formed so as to be positioned lower as the upper edge of the pouch 3 approaches the partition portion 6 in the left-and-right direction. This can prevent the pouch 3 from covering the part in which the patella 83 is located. Thus, this can prevent displacement of the heat generator for knees 1 when the patella 83 moves during exercise bending the knee 8, such as walking or bending and stretching.

Because the heat generator for knees 1 includes the belt 7 for securing the pouch 3 to a leg of a user, the heat generator for knees 1 can be effectively secured to the knee.

### (3) Modification Example

The embodiment above is merely one of various embodiments of the present invention. The embodiment can be modified in various ways according to the design or the like as long as the object of the present invention is achieved. The following describes modification examples of the embodiment. The modification examples described below can be suitably combined for use.

In the embodiment above, the partition portion 6 is continuous in the up-and-down direction, and the upper end and the lower end of the partition portion 6 are connected to the peripheral joint portion 4, thereby enclosing the right section and the left section. However, the partition portion 6 in the present invention does not have to close each section.

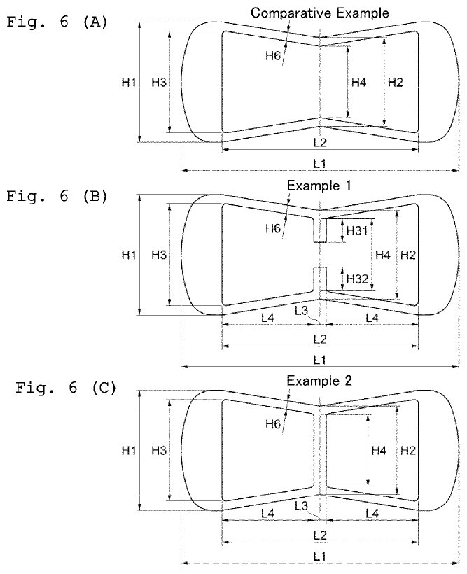

As shown in, for example, FIG. 5(A), the partition portion 6 may be provided such that the total length in the up-and-down direction at the part in which the front sheet 31 and the back sheet 32 are joined is 30% or more, preferably 40% or more, more preferably 60% or more, still more preferably 80% or more, and yet more preferably 100% relative to dimension D1, which is between the intersections of a virtual line (centerline C1) that is in the up-and-down direction and that passes through the center of the width of the partition portion 6 and the inner peripheral edge of the peripheral joint portion 4. For example, as shown in FIG. 5(B) or FIG. 5(C), if the intersections of a virtual line that is in the up-and-down direction and that passes through the center of the width of the partition portion 6 and the inner peripheral edge of the peripheral joint portion 4 are not present, the dimension between the intersections of a virtual line that is in the up-and-down direction and that passes through the center of the width of the partition portion 6 and an extended line of the inner peripheral edge of the peripheral joint portion 4 is determined to be dimension D1.

In the heat generator for knees 1 according to modification example 1, as shown in FIG. 5(A), both ends of the partition portion 6 in the up-and-down direction are apart from the peripheral joint portion 4. However, length D2 of the part joined by the partition portion 6 in the up-and-down direction accounts for 60% or more of D1. Thus, the partition portion 6 is considered to divide the heat generation area 5 into sections in the left-and-right direction.

In this case, the high-temperature region in the left section is located in the center portion of the left section, and the high-temperature region in the right section is located in the center portion of the right section; thus, the high-temperature regions of these sections are apart from each other in the left-and-right direction.

As shown in FIG. 5(B), the partition portion 6 of the heat generator for knees 1 according to modification example 2 includes an upper protrusion 62, a lower protrusion 63, and a middle portion 64 between the upper protrusion 62 and the lower protrusion 63, as portions at which the front sheet 31 and the back sheet 32 are joined. The upper protrusion 62 protrudes downward from the upper intersection, among the intersections of a virtual line (centerline C1) that is in the up-and-down direction and that passes through the center of the width of the partition portion 6 and an extended line of the inner peripheral edge of the peripheral joint portion 4. The lower protrusion 63 protrudes upward from the lower intersection among the intersections. The upper protrusion 62 and the middle portion 64 are apart from each other, and the lower protrusion 63 and the middle portion 64 are also apart from each other. However, the total length of length D3 of the upper protrusion 62 in the up-and-down direction, length D4 of the lower protrusion 63 in the up-and-down direction, and length D2 of the middle portion 64 in the up-and-down direction accounts for 60% or more of dimension D1. Specifically, the partition portion 6 according to this modification example is also considered to divide the heat generation area 5 into sections in the left-and-right direction. Thus, the high-temperature regions of these sections are positioned apart from each other in the left-and-right direction.

As shown in FIG. 5(C), the partition portion 6 of the heat generator for knees 1 according to modification example 3 includes the upper protrusion 62 and the lower protrusion 63 as portions at which the front sheet 31 and the back sheet 32 are joined. The upper protrusion 62 and the lower protrusion 63 are not in contact with each other and are positioned apart from each other. However, the total length of length D3 of the upper protrusion 62 in the up-and-down direction and length D4 of the lower protrusion 63 in the up-and-down direction accounts for 50% or more of dimension D1. Specifically, the partition portion 6 according to this modification example also divides the heat generation area 5 into sections in the left-and-right direction. Thus, the high-temperature regions of these sections are positioned apart from each other in the left-and-right direction.

As in modification examples 1 to 3, when the portion at which the front sheet 31 and the back sheet 32 are joined accounts for 30% or more of dimension D1 as the partition portion 6, the partition portion 6 is considered to divide the heat generation area 5 into sections in the left-and-right direction, and any of these modification examples allows the high-temperature regions within the temperature distribution of the heat generation area 5 to be appropriately applied to both sides of the knee joint.

The following lists modification examples of the embodiment. The modification examples explained below can be suitably combined for use.

In the embodiment above, the partition portion 6 is located in the center portion between the right end and the left end of the heat generation area 5. However, the partition portion 6 may be off from the center portion toward right or left in the present invention.

In the embodiment above, the partition portion 6 is only a single partition portion and divides the heat generation area 5 into two sections. However, two or more partition portions 6 may be provided, and the heat generation area 5 may be divided into three or more sections in the present invention.

In the embodiment above, the upper edge of the heat generation area 5 is formed so as to be positioned lower as the upper edge approaches the partition portion 6 in the left-and-right direction. However, the upper edge of the heat generation area 5 may be parallel to the left-and-right direction in the present invention. Likewise, the upper edge of the pouch 3 may be parallel to the left-and-right direction. The upper edge of the heat generation area 5 and the upper edge of the pouch may not be parallel to each other.

In the embodiment above, the pouch 3 is secured to a leg with the belt 7. However, the pouch 3 may be secured to the knee 8 with an adhesive layer provided to a surface of the pouch 3 without the belt 7 in the present invention.

The heat generator for knees 1 according to the embodiment above is formed symmetrically in the up-and-down direction. However, the heat generator for knees 1 may not be formed symmetrically in the up-and-down direction in the present invention. For example, the upper edge of the pouch may be formed as in embodiment 1, and the lower edge of the pouch may formed in a straight line parallel to the left-and-right direction.

In the present specification, expressions accompanied by “substantially,” such as “substantially rectangular,” may be used. For example, “substantially rectangular” means that an object is substantially rectangular, and the phrase includes not only the meaning of strictly rectangular, but also the meaning that a shape can be generally recognized as rectangular when viewed. The same applies to other expressions with “substantially.”

In the present specification, expressions distinguished by the presence or absence of “part” or “portion” are used, such as “front end portion” and “front end.” For example, an “end portion” means a portion of an area including an “end.” The same applies to other expressions with “part” or “portion.”

### (4) Examples

In Examples 1 and 2, heat generators for knees with a partition portion were produced, and in a Comparative Example, a heat generator for knees with no partition portion was produced. Images of these heat generators were captured by thermography to display the temperature of the heat generating surface at the time of heat generation, and temperature distribution was compared between the heat generators.

In Example 1, as shown in FIG. 6(B), the heat generator for knees had the following dimensions: dimension L1 was 22 cm in the left-and-right direction, dimension H1 at the end portions in the left-and-right direction, among the dimensions in the up-and-down direction, was 9.5 cm, and dimension H2 between the end portions in the center portion, among the dimensions in the up-and-down direction, was 7 cm. The heat generation area had the following dimensions: dimension L2 was 16 cm in the left-and-right direction, dimension L4 of each section in the left-and-right direction was 7.5 cm, dimension H3 at the end portions in the left-and-right direction, among the dimensions in the up-and-down direction, was 8 cm, and dimension H4 between the end portions in the center portion, among the dimensions in the up-and-down direction (corresponding to dimension D1 in the modification example above), was 5.6 cm. The peripheral joint portion had a width H6 of 0.7 cm.

In Example 1, the partition portion was formed in the same manner as in modification example 3, with length H31 of the upper protrusion (corresponding to dimension D3 in the modification example above) being 1.8 cm, length H32 of the lower protrusion (corresponding to dimension D4 in the modification example above) being 1.8 cm, and width L3 of the upper protrusion and the lower protrusion being 1 cm. Specifically, the dimension of the portion at which the front sheet 31 and the back sheet 32 were joined in the partition portion was 1.8+1.8=3.6 cm, which is 64% of dimension H4 (5.6 cm).

In Example 2, as shown in FIG. 6(C), dimension L1, dimension L2, dimension L3, dimension L4, dimension H1, dimension H2, dimension H3, dimension H4, and dimension H6 were the same as those of Example 1. In Example 2, the partition portion was the same as in the embodiment described above.

In a Comparative Example, as shown in FIG. 6(A), dimension L1, dimension L2, dimension H1, dimension H2, dimension H3, dimension H4, and dimension H6 were the same as those in Examples 1 and 2.

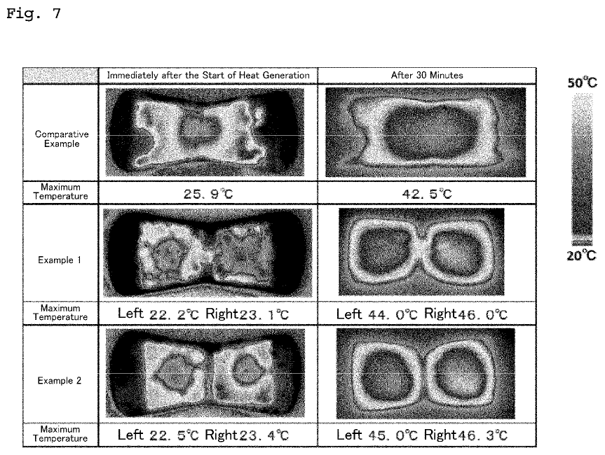

FIG. 7 shows the results of images of Examples 1 and 2 and the Comparative Example captured by thermography.

As is clear from FIG. 7, whereas a high-temperature region (white part) within the temperature region is present in the center portion in the left-and-right direction in the Comparative Example, a high-temperature region (white part) is present in the center portion of each section in Examples 1 and 2, with the high-temperature regions positioned apart from each other in the left-and-right direction. Thus, the high-temperature region can be applied to both sides of the knee joint when the heat generator for knees in Example 1 or 2 is attached.

Additionally, whereas the maximum temperature in the Comparative Example was 42.5° C., the maximum temperature was 44.0° C. in the left section and 46.0° C. in the right section in Example 1, and 45.0° C. in the left section and 46.3° C. in the right section in Example 2. Thus, the temperature of the high-temperature region in Examples 1 and 2 was higher than that in the Comparative Example, allowing the heat generators in Examples 1 and 2 to effectively provide heat to both sides of the knee joint. Moreover, the temperature in the high-temperature region in Example 2 was higher than that in Example 1; i.e., this indicates that a higher proportion of the joined portion relative to the entire partition portion in the lengthwise dimension leads to a higher temperature of the high-temperature regions.

### DESCRIPTION OF THE REFERENCE NUMERALS

1 heat generator for knees
2 heat-generating material
3 pouch
31 front sheet
32 back sheet
4 peripheral joint portion
5 heat generation area
6 partition portion
7 belt
35

1. A heat generator for knees, comprising
a heat-generating material, and
a pouch that is formed from a pair of sheets and that accommodates the heat-generating material inside,
the pouch comprising
a peripheral joint portion that is formed from the pair of sheets that are joined, the pouch having a heat generation area on an inner side of the peripheral joint portion, and
a partition portion that divides the heat generation area into sections in a left-and-right direction by the pair of sheets joined in an up-and-down direction on the inner side of the peripheral joint portion.

2. The heat generator for knees according to claim 1, wherein the partition portion is positioned in a center portion between a right end and a left end of the heat generation area in the left-and-right direction.

3. The heat generator for knees according to claim 1, wherein an upper edge of the heat generation area is formed so as to be positioned lower as the upper edge of the heat generation area approaches the partition portion in the left-and-right direction.

4. The heat generator for knees according to claim 1, wherein an upper edge of the pouch is formed so as to be positioned lower as the upper edge of the pouch approaches the partition portion in the left-and-right direction.

5. The heat generator for knees according to claim 1, further comprising a belt configured to secure the pouch on a user's leg.

6. The heat generator for knees according to claim 2, wherein an upper edge of the heat generation area is formed so as to be positioned lower as the upper edge of the heat generation area approaches the partition portion in the left-and-right direction.

7. The heat generator for knees according to claim 2, wherein an upper edge of the pouch is formed so as to be positioned lower as the upper edge of the pouch approaches the partition portion in the left-and-right direction.

8. The heat generator for knees according to claim 3, wherein an upper edge of the pouch is formed so as to be positioned lower as the upper edge of the pouch approaches the partition portion in the left-and-right direction.

9. The heat generator for knees according to claim 6, wherein an upper edge of the pouch is formed so as to be positioned lower as the upper edge of the pouch approaches the partition portion in the left-and-right direction.

10. The heat generator for knees according to claim 2, further comprising a belt configured to secure the pouch on a user's leg.

11. The heat generator for knees according to claim 3, further comprising a belt configured to secure the pouch on a user's leg.

12. The heat generator for knees according to claim 4, further comprising a belt configured to secure the pouch on a user's leg.

13. The heat generator for knees according to claim 6, further comprising a belt configured to secure the pouch on a user's leg.

14. The heat generator for knees according to claim 7, further comprising a belt configured to secure the pouch on a user's leg.

15. The heat generator for knees according to claim 8, further comprising a belt configured to secure the pouch on a user's leg.

16. The heat generator for knees according to claim 9, further comprising a belt configured to secure the pouch on a user's leg.
